(19) United States
(12) Patent Application Publication
(10) Pub. No.: US 2010/0049211 A1
(43) Pub. Date: Feb. 25, 2010

(54) KIT FOR REMOVING WASTE FROM STRATUM CORNEUM

(75) Inventor: Shu-Bin SHIH, TAIPEI COUNTY (TW)

Correspondence Address: ROSENBERG, KLEIN & LEE, 3458 ELLICOTT CENTER DRIVE-SUITE 101, ELLICOTT CITY, MD, 21043, US

(73) Assignee: FENG-WEN TENG, YUNLIN COUNTY (TW)

(21) Appl. No.: 12/211,389

(22) Filed: Sep. 16, 2008

(30) Foreign Application Priority Data
Aug. 20, 2008 (CN) 200820208011.9

(51) Int. Cl. A61B 17/50

(52) U.S. Cl. 606/131

(57) ABSTRACT

A kit for removing stratum corneum includes a grip body and a ceramic plate. The grip body has an exterior part defining a gripping section, an interior part defining a reception chamber and a clamping structure disposed around the periphery of the reception chamber. The ceramic plate is clamped by the clamping structure in such a manner that the ceramic plate is disposed securely above the reception chamber. The ceramic plate has a plurality of through holes communicating spatially with the reception chamber and one protrusive element projecting outward from a certain peripheral portion of a respective one of the through holes away from the reception chamber while the protrusive element cooperates with a remaining peripheral portion of the respective one of the through holes to define a gap therebetween. Scrubbing action of the stratum corneum of a living thing against the protrusive element results in waste, which falls into the reception chamber through the gap in the ceramic plate.

This application claims the benefit of China Utility Model Application Serial NO. 200820208011.9, filed on Aug. 20, 2008, the subject matter of which is incorporated herein by reference.

### BACKGROUND OF THE INVENTION

1. Field of the Invention

The present invention relates to a kit for removing waste from stratum corneum of a living thing, more particularly to ones in which a ceramic plate is utilized for removing the waste from the stratum corneum.

2. Description of the Prior Art

Human being flesh is covered by an epidermis (colloquially called skin) to protect the same from being infected by external environment. However, the stratum corneum (colloquially horny layer) of the epidermis is generally hard and thorny since the same is made up of dead cells that gradually break apart into waste or dirt. In one way, the stratum corneum is beneficial to human because it protects the stratum germinetrium (cell producing layer). In the other way, it looks ugly since its external surface is rough and horny. Modern people, especially beauty-adoring ladies, are in the habit of removing waste from the stratum corneum in order to enhance the beauty thereof.

Generally speaking, a pumice stone or floatstone is used to scrub against the stratum corneum of a human being for removing waste therefrom. After scrubbing the stratum corneum for a period of time, the environment is scattered with the waste resulting therefrom and thus creating an unhygienic condition for living. Moreover, the floatstone itself is formed with a plurality of clearances on its external surface so that the waste adhering therein is contaminated in the long run, thereby causing disgust and discomfort to the person using the same floatstone.

Some waste-removing kits have been invented by using metals. These kits are not formed with through holes for passage of the waste resulting from scrubbing action of the stratum corneum against the kits such that the same kits are mostly contaminated in long-term use, though the contamination is not visible with the naked eyes.

Presently, the technology for treating a ceramic plate is quite mature and the tangibility becomes more and more clear. Since ceramic has antiseptic property, the object of the present invention is to implement a ceramic plate in the kit for removing waste from the stratum corneum, thereby avoiding the above-mentioned drawbacks encountered during use of the conventional kits for removing waste of the stratum corneum.

### SUMMARY OF THE INVENTION

The object of the present invention is to provide a kit for removing waste from the stratum corneum, in which a ceramic plate, possessing the antiseptic property, is utilized for removing the waste from the stratum corneum.

The other object of the present invention is to provide a kit for removing waste of the stratum corneum, wherein, the kit is easy to be gripped by the user and the waste resulting from scrubbing the stratum corneum against the kit can be collected within a reception chamber of the kit, thereby keeping the environment free from pollution.

In one aspect of the present invention, a kit for removing waste from stratum corneum is provided to include a grip body and a ceramic plate. The grip body has an exterior part defining a gripping section, an interior part defining a reception chamber and a clamping structure disposed around the periphery of the reception chamber. The ceramic plate is clamped by the clamping structure in such a manner that the ceramic plate is disposed securely above the reception chamber. The ceramic plate has with a plurality of through holes communicating spatially with the reception chamber and one protrusive element projecting outward from a certain peripheral portion of a respective one of the through holes away from the reception chamber while the protrusive element cooperates with a remaining peripheral portion of the respective one of the through holes to define a gap therebetween.

Scrubbing action of the stratum corneum of a living thing against the protrusive element results in waste, which falls into the reception chamber through the gap in the ceramic plate, thereby keeping the environment in a hygienic condition.

The kit of the present invention further includes a cover member for covering onto the grip body so as to enclose the ceramic plate between the cover member and the grip body, thereby providing an additional aesthetic beauty to the kit.

### BRIEF DESCRIPTION OF THE DRAWINGS

Other features and advantages of this invention will become more apparent in the following detailed description of the preferred embodiment of this invention, with reference to the accompanying drawings, in which:

FIG. 1 is a perspective view of a kit of the present invention without a cover member for removing waste from stratum corneum of a living thing;

FIG. 2 is an exploded view of the kit of the present invention for removing waste from stratum corneum of a living thing;

FIG. 3 is a perspective view of the kit of the present invention with the cover member and the kit is used for removing waste from stratum corneum of a living thing; and

FIG. 4 shows a modified embodiment of a kit of the present invention for removing waste from stratum corneum of a living thing.

### DETAILED DESCRIPTION OF THE PREFERRED EMBODIMENTS

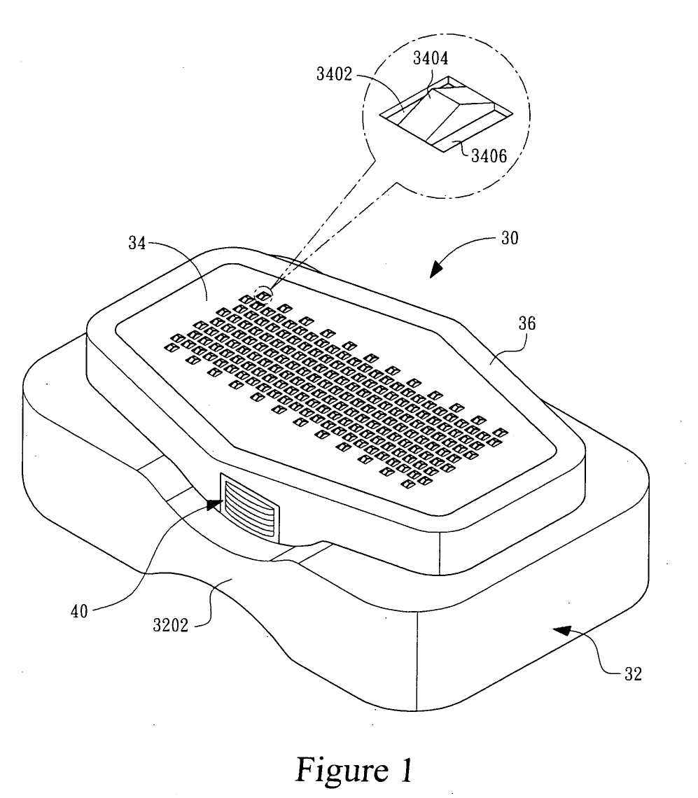

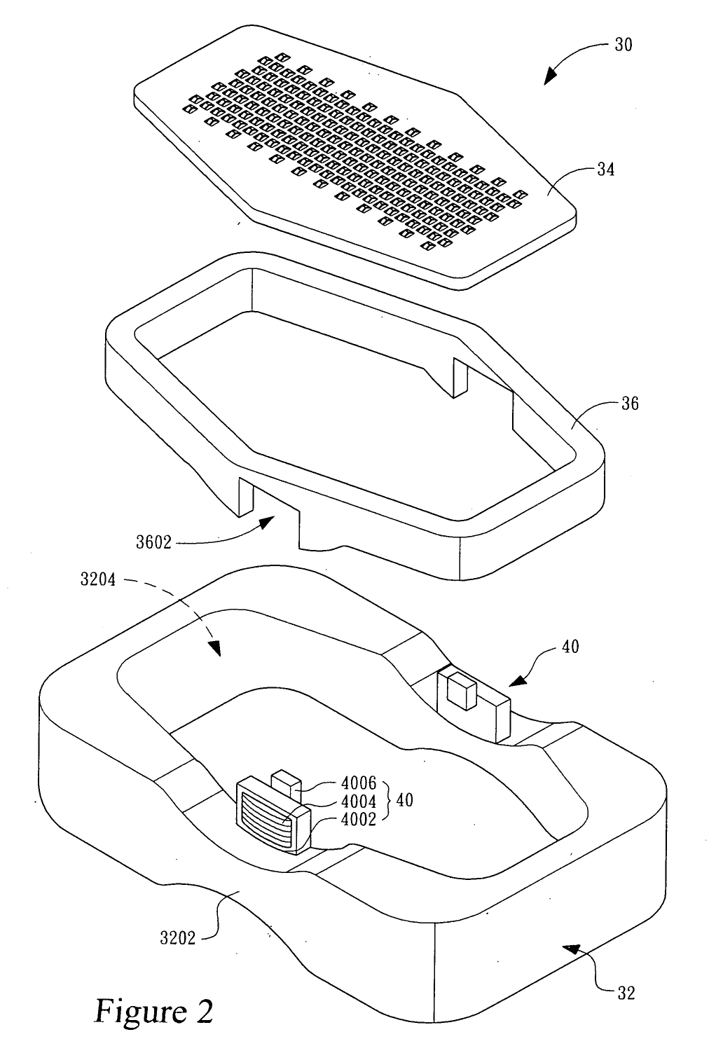

Referring to FIGS. 1 and 2, wherein FIG. 1 is a perspective view of a kit 30 of the present invention without a cover member for removing waste from stratum corneum of a living thing (not shown) while FIG. 2 is an exploded view of the kit 30 of the present invention for removing the waste from the stratum corneum. As illustrated, the kit 30 includes a grip body 32, a ceramic plate 34 and a hollow frame 36.

The grip body 32 has an exterior part defining a gripping section 3202, an interior part defining a reception chamber 3204 and a clamping structure 40 disposed around the periphery of the reception chamber 3204;

The ceramic plate 34 is clamped by the clamping structure 40 in such a manner that the ceramic plate 34 is disposed securely above the reception chamber 3204. The ceramic plate 34 has with a plurality of through holes 3402 communicating spatially with the reception chamber 3204 and a plurality of protrusive elements 3404. Each of the protrusive elements 3404 projects outward from a certain peripheral portion of a respective one of the through holes 3402 away from the reception chamber 3204 while the protrusive element 3404 cooperates with a remaining peripheral portion of the respective one of the through holes to define a gap 3406 therebetween.

Under this condition, when a man scrub the stratum corneum on his foot or hand against the protrusive elements 3404 in the ceramic plate 34, waste is resulted and falls into the reception chamber 3204 through the gaps 3406 in the ceramic plate 34. Since the waste is collected in the reception chamber 3204, the surrounding environment is free from contamination, thereby providing a hygienic condition suitable for dwelling.

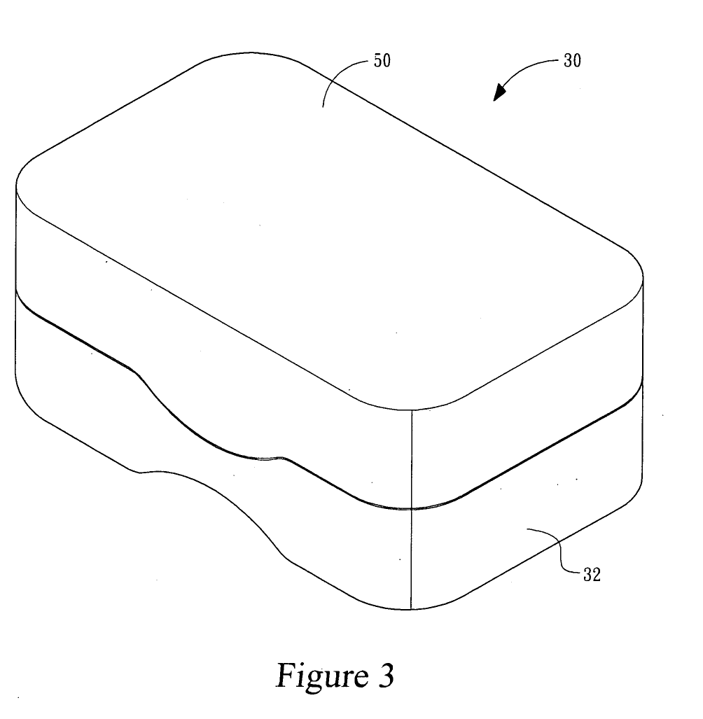

Referring to FIG. 3, the kit 30 of the present invention further includes the cover member 50 for covering onto the grip body 32 so as to enclose or conceal the ceramic plate 34 between the cover member 50 and the grip body 32, thereby providing additional aesthetic beauty to the kit 30.

Referring again to FIG. 2, the kit 30 of the present invention further includes a hollow frame 36 disposed on the grip body 32 and around the ceramic plate 34 for holding the ceramic plate 34. In this embodiment, the clamping structure 40 includes two fixing sections 4002, two resilient sections 4004 and two retention sections 4006.

Each fixing section 4002 is attached securely on the peripheral portion of the reception chamber 3204 (i.e. on the gripping section 3202 of the grip body 32). Each resilient section 4004 is mounted on an outer side of the fixing section 4002 and exposes to an exterior of the reception chamber 3204. Each retention section 4006 is mounted on an inner side of the fixing section 4002 for resiliently retaining the ceramic plate 34 above the reception chamber 3204. Preferably, the hollow frame 36 has two downward recesses 3602 for seating above the fixing sections 4002 in such a manner that the retention sections 4006 are disposed inward of the hollow frame 36 to abut and support the ceramic plate 34 from underneath. When it is desired to remove the ceramic plate 34, two fingers of a user must squeeze the resilient sections 4004 simultaneously from an outer side thereof, which, in turn, causes disengagement of the ceramic plate 34 from the clamping structure 40 (hence the hollow frame 36). At this time, the user can clean the waste off the reception chamber 3204.

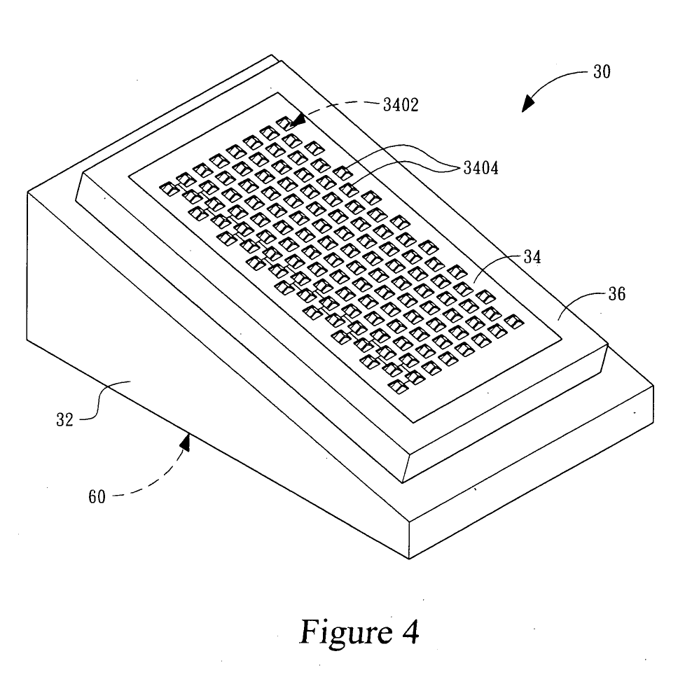

FIG. 4 shows a modified embodiment of the kit 30 of the present invention and has the structure similar to the previous embodiment, except in that the exterior part of the grip body 32 is formed with a flat bottom for disposing on a horizontal support surface 60 while the interior part is formed with an inclined surface for supporting the ceramic plate 34 thereon. When thus arranged, one needs not hold the kit 30 of the present invention to scrub the protrusive elements 3404 (see FIG. 1) against the stratum corneum. Instead, he can stand up and scrub his stratum corneum against the protrusive elements 3404 easily in the forward, rearward or sidewise directions such that the waste resulting thereby falls into the reception chamber 3204 via the gaps 3406 in the ceramic plate 34.

Note that the ceramic plate 34 employed in the kit 30 of the present invention has a plurality of through holes 3402 and gaps 3406, in addition to possessing antiseptic property. The waste resulting from the scrubbing action of the stratum corneum against the protrusive elements in the ceramic plate 34 is collected in the reception chamber 3204. Since no waste is accumulated on the ceramic plate 34, no contamination is resulted, thereby providing a hygienic environment for dwelling.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A kit for removing waste from stratum corneum comprising:
a grip body 32 having an exterior part defining a gripping section 3202, an interior part defining a reception chamber 3204 and a clamping structure 40 disposed around the periphery of said reception chamber 3204;
a ceramic plate 34 being clamped by said clamping structure 40 in such a manner that said ceramic plate 34 is disposed above said reception chamber 3204, said ceramic plate 34 having with a plurality of through holes 3402 communicating spatially with said reception chamber 3204 and at least one protrusive element 3404 projecting outward from a certain peripheral portion of a respective one of said through holes 3402 away from said reception chamber 3204 while said protrusive element 3404 cooperates with a remaining peripheral portion of said respective one of said through holes to define a gap 3406 therebetween;
wherein, scrubbing action of the stratum corneum of a living thing against said protrusive element 3404 results in waste, which falls into said reception chamber 3204 through said gap in said ceramic plate 34.

2. The kit according to claim 1, further comprising a cover member 50 for covering onto said grip body 32 so as to enclose said ceramic plate 34 between said cover member 50 and said grip body 32.

3. The kit according to claim 1, wherein said clamping structure 40 includes a fixing section 4002 attached securely on the periphery of said reception chamber 3204, a resilient section 4004 mounted on an outer side of said fixing section 4002 and exposing to an exterior of said reception chamber 3204 and a retention section 4006 mounted on an inner side of said fixing section 4002 for resiliently retaining said ceramic plate 34 above said reception chamber 3204.

4. The kit according to claim 3, further comprising a hollow frame 36 disposed on said grip body and around said ceramic plate 34 for holding said ceramic plate 34, said hollow frame 36 having a downward recess 3602 for seating above said fixing section 4002 in such a manner that said retention section 4006 is disposed inward of said hollow frame 36 to abut and support said ceramic plate 34 from underneath.

5. The kit according to claim 1, wherein said exterior part of said grip body 32 is formed with a flat bottom for disposing on a horizontal support surface while said interior part is formed with an inclined surface for supporting said ceramic plate 34 thereon.
